(19) United States
(12) Patent Application Publication
(10) Pub. No.: US 2025/0268823 A1
(43) Pub. Date: Aug. 28, 2025

(54) Aqueous Quetiapine Solutions

(71) Applicant: RELIIS LTD, West Perth (AU)

(72) Inventors: Antony Condina, Sydney, New South Wales (AU); Steven Anthony Rochford Webb, Sydney, New South Wales (AU); Edward Litton, Sydney, New South Wales (AU)

(21) Appl. No.: 18/858,223

(22) PCT Filed: Apr. 19, 2023
(86) PCT No.: PCT/AU2023/050316

§ 371 Date: Oct. 18, 2024

(30) Foreign Application Priority Data
Apr. 19, 2022 (AU) 2022901032

(51) Int. Cl. A61K 9/08; A61K 9/00; A61K 31/554; A61K 47/10; A61K 47/12; A61K 47/18

(52) U.S. Cl. CPC A61K 9/08; A61K 9/0019; A61K 31/554; A61K 47/10; A61K 47/12; A61K 47/183

(57) ABSTRACT

There is provided an aqueous solution comprising a quetiapine salt and either or both of a benzoic acid salt and a citric acid salt. Preferably the concentration of the quetiapine salt is at least 0.1 mg/ml to about 5 mg/ml. The solutions are useful for the prevention and treatment of delirium, particularly in an intensive care setting.

### TECHNICAL FIELD

The technology relates to aqueous solutions of quetiapine fumarate for intravenous administration. The solutions are useful for the treatment of delirium in a hospital setting.

### RELATED APPLICATION

This application claims the benefit of Australian Provisional Patent Application No. 2022901032 filed 19 Apr. 2022, which is herein incorporated by reference in its entirety.

### BACKGROUND

Quetiapine (2-(2-(4-(dibenzo[b,f][1,4]thiazepin-11-yl)piperazin-1-yl)ethoxy)ethanol) is used in the treatment of severe mental illness. Quetiapine is a dibenzothiazepine derivative with a relatively broad receptor binding profile. It has major affinity to cerebral serotonergic (5-HT_{2A}), histaminergic (H1), and dopaminergic D₁ and D₂ receptors, moderate affinity to α1- and α2-adrenergic receptors, and minor affinity to muscarinergic M1 receptors. This receptor avidity profile with relatively higher affinity for the 5-HT_{2A} receptor compared to the D₂ receptor is considered to be, at least in part responsible for the antipsychotic characteristics and low incidence of extrapyramidal side-effects of quetiapine.

Quetiapine has demonstrated robust efficacy for the treatment of cognitive, anxious-depressive, and aggressive symptoms in schizophrenia. Quetiapine also has proven efficacy and tolerability in the treatment of moderate to severe manic episodes, and in the treatment of juveniles with oppositional-defiant or conduct disorders, and in the treatment of the geriatric population with dementia. Data indicate that quetiapine is also effective in the treatment of bipolar depressive symptoms without increasing the risk of triggering manic episodes, and in the treatment of borderline personality disorder.

Quetiapine has an acceptable efficacy in a number of off-label indications that include obsessive-compulsive disorder, post-traumatic stress disorder, restless legs syndrome, autism, alcoholism, depression, Tourette syndrome and in the treatment of delirium in ICU (intensive care unit) patients.

Patients in emergency departments, in hospital wards or in an ICU can suffer from delirium which is distressing to the patient and their family and is associated with increased duration of stay, mortality, and subsequent cognitive impairment. The optimal management of delirium requires a calm environment, sleep hygiene and correction of underlying factors (for example, infection). This can be a challenge in the ICU, and drug therapy, commonly haloperidol, clonidine, benzodiazepines, dexmedetomidine, olanzapine or propofol, is often used. Quetiapine has been used in the ICU for the treatment of delirium.

However, the solubility of quetiapine fumarate, the most commonly used quetiapine salt is about 0.04 mg/ml in water. Consequently, quetiapine is conventionally formulated as immediate or modified-release tablets. Oral suspensions are not commercially available but published protocols for 10 and 40 mg/ml compounded suspensions of quetiapine are available and involve suspending ground tablets in aqueous vehicles (e.g. Ora-Sweet and Ora-Blend). This is problematic as quetiapine fumarate has poor oral bioavailability (9%) due to extensive hepatic first-pass metabolism.

The present inventors have developed aqueous quetiapine fumarate solutions for intravenous administration.

### SUMMARY

In a first aspect, there is provided an aqueous solution comprising a quetiapine salt and either or both of a benzoic acid salt and a citric acid salt. Preferably, the concentration of the quetiapine salt is at least about 0.1 mg/ml.

In an embodiment the concentration of the quetiapine salt is at least 0.1 mg/ml to about 5 mg/ml.

In one embodiment the quetiapine salt is selected from the group comprising quetiapine fumarate, quetiapine hemifumarate, quetiapine hydrobromide, quetiapine hydrochloride, quetiapine malate, quetiapine tartrate, quetiapine oxalate, quetiapine succinate, quetiapine benzoate, quetiapine sulfonate, quetiapine naphthalene-sulfonate, quetiapine naphsylate, and quetiapine tosylate. Preferably the quetiapine salt is quetiapine fumarate.

In one embodiment the quetiapine is not conjugated to an amino acid or fatty acid.

The benzoic acid salt may be selected from the group comprising sodium benzoate, potassium benzoate, calcium benzoate, magnesium benzoate, lithium benzoate, and ammonium benzoate. Preferably the benzoic acid salt is sodium benzoate.

The citric acid salt may be selected from calcium citrate, sodium citrate, and potassium citrate. Preferably the citric acid salt is sodium citrate.

In some embodiments the solution further comprises one or more of an amino acid, a glycol and a pharmaceutically acceptable organic acid.

The amino acid may be an L-amino acid selected from the group comprising of alanine, arginine, asparagine, aspartate, cysteine, glutamate, glutamine, glycine, histidine, hydroxyproline, isoleucine, leucine, lysine, methionine, phenylalanine, proline, pyroglutamate, serine, threonine, tryptophan, tyrosine, and valine. Preferably the amino acid is L-arginine.

The pharmaceutically acceptable organic acid may be one or more of benzoic acid, citric acid, oxalic acid, malic acid, tartaric acid, lactic acid, ascorbic acid, and glucuronic acid, preferably benzoic acid.

The glycol may be selected from propylene glycol, ethylene glycol, polypropylene glycol, and polyethylene glycol, preferably the glycol is propylene glycol.

The solutions may further comprise one or more pharmaceutically acceptable carriers, diluents or excipients.

In one embodiment the solutions are formulated for intravenous use.

In one embodiment the solutions have a pH in the range of 5-7, preferably the pH is about 6.1, 6.2, 6.3, 6.4, 6.5, 6.6, 6.7, 6,8, 6.9 or about 7.0.

In a second aspect there is provided a method of preventing, treating or ameliorating at least one symptom of delirium in a subject the method comprising administering to the subject a therapeutically effective amount of the quetiapine solution of the first aspect.

In one embodiment the delirium is characterised by a state of reduced cognition and responsiveness; a state of hypervigilance, agitation and confusion; or a fluctuation between the states.

In an embodiment the subject is critically ill, for example a patient in intensive care, in a psychiatric facility, an emergency department, or is admitted to a hospital ward.

In one embodiment the administering step comprises intravenous administration of the quetiapine solution.

In one embodiment the administering step comprises subcutaneous administration of the quetiapine solution.

### DEFINITIONS

Throughout this specification, unless the context requires otherwise, the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps. The term ‘consisting of’ means ‘consisting only of’, that is, including and limited to the stated element(s), integer(s) or step(s), and excluding any other element(s), integer(s) or step(s). The term ‘consisting essentially of’ means the inclusion of the stated element(s), integer(s) or step(s), but other element(s), integer(s) or step(s) that do not materially alter or contribute to the working of the invention may also be included.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (i.e., at least one) of the grammatical object of the article. By way of example, reference to ‘an element’ means one element, or more than one element.

In the context of the present specification the term ‘about’ means that reference to a figure or value is not to be taken as an absolute figure or value, but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

The term ‘pharmaceutically acceptable salt’ refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. For a review on suitable salts, see Handbook of Pharmaceutical Salts: Properties, Selection, and Use by Stahl and Wermuth (Wiley-VCH, 2002). Methods for making pharmaceutically acceptable salts of compounds of the invention are known to one of skill in the art. The salts can be prepared in situ during the final isolation and purification of the compounds of the invention, or separately by reacting the free base function with a suitable organic acid. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, methanesulfonic, salicylic, p-hydroxybenzoic, phenylacetic, mandelic, ambonic, pamoic, pantothenic, sulfanilic, cyclohexylaminosulfonic, stearic, algenic, β-hydroxybutyric, galactaric, fumaric and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, suitable pharmaceutically acceptable base addition salts of the compounds of the present invention include organic salts made from N,N′-dibenzylethylenediamine, chloroprocaine, choline, diethanolamine, ethylenediamine, meglumine (N-methylglucamine), procaine, ammonium salts, quaternary salts such as tetramethylammonium salt, amino acid addition salts such as salts with glycine and arginine. In the case of compounds that are solids, it will be understood by those skilled in the art that the inventive compounds, agents and salts may exist in different crystalline or polymorphic forms, all of which are intended to be within the scope of the present invention and specified formulae.

The terms ‘treating’, ‘treatment’ and ‘therapy’ are used herein to refer to curative therapy, prophylactic therapy, palliative therapy and preventative therapy. Thus, in the context of the present disclosure the term ‘treating’ encompasses curing, ameliorating or tempering the severity of a medical condition or one or more of its associated symptoms.

The terms ‘therapeutically effective amount’ or ‘pharmacologically effective amount’ or ‘effective amount’ refer to an amount of an agent sufficient to produce a desired therapeutic or pharmacological effect in the subject being treated. The terms are synonymous and are intended to qualify the amount of each agent that will achieve the goal of improvement in disease severity and/or the frequency of incidence over treatment of each agent by itself while preferably avoiding or minimising adverse side effects, including side effects typically associated with other therapies. Those skilled in the art can determine an effective dose using information and routine methods known in the art.

A ‘pharmaceutical carrier, diluent or excipient’ includes, but is not limited to, any physiological buffered (i.e., about pH 6.0 to 7.4) medium comprising a suitable water-soluble organic carrier, conventional solvents, dispersion media, fillers, solid carriers, coatings, antibacterial and antifungal agents, isotonic and absorption delaying agents. Suitable water-soluble organic carriers include, but are not limited to, saline, dextrose, corn oil, dimethylsulfoxide, and gelatin capsules. Other conventional additives include lactose, mannitol, corn starch, potato starch, binders such as microcrystalline cellulose, cellulose derivatives such as hydroxypropylmethylcellulose, acacia, gelatins, disintegrators such as sodium carboxymethylcellulose, and lubricants such as talc or magnesium stearate.

‘Subject’ includes any human or non-human mammal. Thus, in addition to being useful for human treatment, the compounds of the present invention may also be useful for veterinary treatment of mammals, including companion animals and farm animals, such as, but not limited to dogs, cats, horses, cows, sheep, and pigs. In preferred embodiments the subject is a human.

In the context of this specification the term ‘administering’ and variations of that term including ‘administer’ and ‘administration’, includes contacting, applying, delivering or providing a compound or composition of the invention to a subject by any appropriate means.

The term ‘critically ill’ or ‘critical illness’ refers to a medical condition in which a patient, because of major surgery or severe illness, requires immediate intensive medical support in order to survive or recover, independent of the underlying condition.

### DESCRIPTION OF EMBODIMENTS

The invention relates to aqueous solutions of quetiapine comprising either or both of a benzoic acid salt and a citric acid salt (e.g. sodium benzoate or sodium citrate, respectively) and optionally one or more of sodium chloride, benzoic acid, L-arginine and propylene glycol. Preferably the quetiapine is quetiapine fumarate. Preferably the quetiapine concentration is 0.1 mg/ml to about 5 mg/ml.

The aqueous solutions of quetiapine described herein have a number of advantages compared to oral quetiapine. Those advantages may include one or more of the following:
Increased quetiapine solubility, for example up to at least about 5 mg/ml.
Reduced interindividual variability in plasma concentrations compared to oral quetiapine.
Self-preserved formula.
The solutions have a near neutral pH.
Rapid solubilisation and method of manufacture.
Increased bioavailability.
Improved side-effect profile.
Ability to quickly and accurately titrate dose thereby allowing individualised treatment.
Precisely control the plasma levels of quetiapine after administration.
The solutions can be administered to patients in whom enteral administration is not possible (for example due to one or more of refusal, absence of a safe and effective swallow, and absence of an enteric feeding tube).
IV administration of the solutions will result in a rapid and predictable onset of action.

The quetiapine solutions described herein may comprise a pharmaceutically effective amount of quetiapine, in association with one or more pharmaceutically acceptable excipients including carriers, vehicles and diluents. The term “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solution for oral, parenteral, intradermal, subcutaneous, or topical application. Excipients can include, by way of illustration and not limitation, diluents, wetting agents, polymers, lubricants, stabilizers, and substances added to mask or counteract a disagreeable taste or odor, flavors, dyes, fragrances, and substances added to improve appearance of the composition. Acceptable excipients include (but are not limited to) stearic acid, magnesium stearate, sodium and calcium salts of phosphoric and sulfuric acids, magnesium carbonate, dextrin, mannitol, sorbitol, lactose, sucrose, starches, gelatin, polymers such as polyvinyl-pyrrolidone, polyvinyl alcohol, and polyethylene glycols, and other pharmaceutically acceptable materials. Examples of excipients and their use is described in Remington's Pharmaceutical Sciences, 20th Edition (Lippincott Williams & Wilkins, 2000). The choice of excipient will to a large extent depend on factors such as the mode of administration, the effect of the excipient on solubility and stability, and the nature of the dosage form.

The quetiapine solutions described herein may be formulated for oral, injectable, parenteral, subcutaneous, intravenous, or intramuscular delivery. The quetiapine solutions of the invention may also be formulated for sustained delivery.

The solutions comprise quetiapine or a pharmaceutically acceptable salt or hydrate thereof. Suitable quetiapine salts include quetiapine fumarate, quetiapine hemifumarate, quetiapine hydrobromide, quetiapine hydrochloride, quetiapine malate, quetiapine tartrate, quetiapine oxalate, quetiapine succinate, quetiapine benzoate, quetiapine sulfonate, quetiapine naphthalene-sulfonate, quetiapine naphsylate, or quetiapine tosylate.

Preferably the quetiapine is quetiapine fumarate.

As noted above the solubility of quetiapine fumarate in water is about 0.04 mg/ml, i.e. effectively insoluble in water. The present inventors have found that combining a quetiapine salt such as quetiapine fumarate with a salt of a citric or benzoic acid results in an aqueous solution of quetiapine solution in which the concentration of dissolved quetiapine is at least about 0.1 mg/ml to about 5 mg/ml.

In some embodiments the quetiapine concentration in the solution is about 0.1 mg/ml, about 0.25 mg/ml, about 0.5 mg/ml, about 0.75 mg/ml, about 1 mg/ml, about 1.25 mg/ml, about 1.5 mg/ml, about 1.75 mg/ml, about 2 mg/ml, about 2.25 mg/ml, about 2.5 mg/ml, about 2.75 mg/ml, about 3 mg/ml, about 3.25 mg/ml, about 3.5 mg/ml, about 3.75 mg/ml, about 4 mg/ml, about 4.25 mg/ml, about 4.5 mg/ml, about 4.75 mg/ml, or about 5 mg/ml.

In one embodiment the quetiapine is not conjugated or otherwise bound to an amino acid, fatty acid or any other moiety.

The solutions comprise either or both of a citrate acid salt or a benzoic acid salt. It is envisaged that any pharmaceutically acceptable salt of citrate acid salt or a benzoic acid salt may be used.

Suitable citric acid salts include calcium citrate, sodium citrate, potassium citrate, magnesium citrate. calcium citrate tetrahydrate, potassium citrate tribasic, monosodium citrate, disodium citrate, trisodium citrate, trisodium citrate dihydrate, trisodium citrate pentahydrate, or a combination thereof.

In one embodiment the citric acid salt is calcium citrate, sodium citrate, or potassium citrate. In a preferred embodiment the citric acid salt is sodium citrate.

Suitable benzoic acid salts include sodium benzoate, potassium benzoate, calcium benzoate, magnesium benzoate, lithium benzoate, ammonium benzoate, 2-(dodecyloxy)benzoate, 2-[(4-[2-[4-(4-chlorobenzyl)-1-piperazinyl]ethoxy]phenyl)sulfanyl]benzoic acid maleate salt, 4-sulfobenzoic acid potassium salt, or a combination thereof.

In one embodiment the benzoic acid salt is sodium benzoate, potassium benzoate, or calcium benzoate. In a preferred embodiment the benzoic acid salt is sodium benzoate.

The concentration of the benzoic acid salt in the formulation ranges from about 5 mg/ml to about 150 mg/ml, for example about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 75 mg/ml, about 70 mg/ml, about 75 mg/ml, about 80 mg/ml, about 85 mg/ml, about 90 mg/ml, about 95 mg/ml, about 100 mg/ml, about 105 mg/ml, about 110 mg/ml, about 115 mg/ml, about 120 mg/ml, about 125 mg/ml, about 130 mg/ml, about 140 mg/ml, about 145 mg/ml, about 150 mg/ml, or more than about 150 mg/ml.

In one embodiment the concentration of the benzoic acid salt in the formulation ranges from about 30 mg/ml to about 50 mg/ml, for example about 40 mg/ml.

The concentration of the citric acid salt in the formulation ranges from about 0.1 mg/ml to about 15 mg/ml, for example about 0.1 mg/ml, 0.25 mg/ml, about 0.5 mg/ml, about 0.75 mg/ml, about 1 mg/ml, about 1.25 mg/ml, about 1.5 mg/ml, about 1.75 mg/ml, about 2 mg/ml, about 2.25 mg/ml, about 2.5 mg/ml, about 2.75 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 10.5 mg/ml, about 11 mg/ml, about 11.5 mg/ml, about 12 mg/ml, about 12.5 mg/ml, about 13 mg/ml, about 13.5 mg/ml, about 14 mg/ml, about 14.5 mg/ml, about 15 mg/ml, or more than about 15 mg/ml.

In one embodiment the concentration of the citric acid salt in the solution ranges from about 0.1 mg/ml to 2 mg/ml, for example about 0.75 mg/ml.

In some embodiments the solutions further comprise one or more of an organic acid such as benzoic acid, a salt such as sodium chloride, and an amino acid such as L-arginine.

In one embodiment the organic acid is one or more of benzoic acid, citric acid, oxalic acid, malic acid, tartaric acid, lactic acid, ascorbic acid, or glucuronic acid. In one embodiment the organic acid is benzoic acid.

In some embodiments the solutions further comprise a glycol such as propylene glycol, ethylene glycol, polypropylene glycol, polyethylene glycol and the like. In one embodiment the solution further comprises propylene glycol.

In some embodiments the solutions further comprise an amino acid. For example, an L-amino acid selected from the croup comprising alanine, arginine, asparagine, aspartate, cysteine, glutamate, glutamine, glycine, histidine, hydroxyproline, isoleucine, leucine, lysine, methionine, phenylalanine, proline, pyroglutamate, serine, threonine, tryptophan, tyrosine, and valine. Preferably the amino acid is L-arginine.

The solutions may further comprise one or more pharmaceutically acceptable carriers, diluents or excipients. The solutions are preferably sterile, isotonic and have a pH within the range pH 3-8, preferably pH 5-7 or pH 6.7, for example pH is about 6.1, 6.2, 6.3, 6.4, 6.5, 6.6, 6.7, 6,8, 6.9 or about 7.0. The solutions can contain one or more buffers as part of a buffer system, however, the concentration of buffers is preferably kept as low as possible. Buffer stressing studies may be carried out to select the minimal buffer amount needed to safely maintain the desired pH range.

In another embodiment, in the solutions comprising the quetiapine salt, for example quetiapine fumarate, the quetiapine salt has improved water solubility over the same quetiapine salt in the absence of the citric acid salt or benzoic acid salt. This may provide better bioavailability of quetiapine referring to a higher area under the curve (AUC) or having higher circulating plasma concentrations.

In another embodiment, the solutions comprising the quetiapine salt, for example quetiapine fumarate, the quetiapine salt has improved bioavailability, such as a higher area under the curve (AUC) or higher circulating plasma concentration, compared to the same quetiapine salt in a solution without citric acid salt or benzoic acid salt.

In another embodiment, the solutions described herein provide:
a. maximum plasma concentration (C max) value of quetiapine that is higher than the C max value produced by oral quetiapine when administered at a therapeutically equivalent dose;
b. in generating an AUC value of quetiapine that is higher than the AUC value produced by oral quetiapine when administered at a therapeutically equivalent dose;
c. both a C max and an AUC value of quetiapine that is higher than the C max and AUC values produced by oral quetiapine when administered at a therapeutically equivalent dose;
d. a time after administration at which C max occurs (T max) value of quetiapine and their combination that is longer than the T max value produced by oral quetiapine when administered at a therapeutically equivalent dose; and/or
e. a T max value of quetiapine that is similar to the T max value produced by oral quetiapine, when administered at therapeutically equivalent dose.

In one embodiment, the term “therapeutically equivalent” refers to circumstances where the quetiapine solution described herein is administered in an amount which gives rise to the same therapeutic effect as a specified amount of a conventional oral quetiapine formulation. It is routine for those skilled in the art to determine therapeutically equivalent amounts or dosages.

In another embodiment, the terms “therapeutically effective”, or “therapeutic effect”, refers to that amount of amount quetiapine in the aqueous solution, which will relieve to some extent one or more of the symptoms of the disorder or disease being treated. In another embodiment, the term “therapeutically effective dose” refers to the amount quetiapine in the aqueous solution, when administered to an individual is effective to at least partially treat a disorder, disease or condition from which the individual is suffering, or to at least partially ameliorate a symptom of such disorder, disease or condition. As is understood in the art, the therapeutically effective amount of quetiapine in the aqueous solution will depend at least in part upon, the mode of administration, any carrier or vehicle employed, the specific disorder or condition, other medications taken by the individual and the specific characteristics of the individual to whom the compound is to be administered (age, weight, condition, sex, etc.).

In another embodiment, the therapeutically equivalent doses would have reduced interindividual variability, for example due to increased bioavailability.

In yet another embodiment, the quetiapine solutions described herein would have increased bioavailability over oral quetiapine. This allows for administration of a lower dose with equal or improved therapeutic effect, but with fewer and/or less severe side-effects when compared to oral quetiapine, thereby improving the safety and/or tolerability profile of the drug. This also provides a clinician with the ability to finely tune the dose of quetiapine to an individual.

In embodiments where the solutions are formulated for oral delivery the solutions may contain conventional additives, such sorbitol, methyl cellulose, glucose syrup, gelatin, hydroxyethyl cellulose, carboxymethyl cellulose, aluminium stearate gel or hydrogenated edible fats, emulsifying agents, for example, lecithin, sorbitan monooleate, or acacia; preservatives, for example, methyl or propyl p-hydroxybenzoate or sorbic acid; and, if desired, conventional flavoring or coloring agents.

For parenteral administration, including intravenous, intramuscular, subcutaneous, or intraperitoneal administration, the quetiapine is formulated as an injectable solution.

In some embodiments the amount of quetiapine that is administered and the dosage regimen for treating a disease with the solutions of the present invention depends on a variety of factors, including the age, weight, sex, and medical condition of the subject, the severity of the disease, the route and frequency of administration, the particular compound employed, as well as the pharmacokinetic properties (e.g., adsorption, distribution, metabolism, excretion) of the individual treated, and thus may vary widely. Such treatments may be administered as often as necessary and for the period of time judged necessary by the treating physician. One of skill in the art will appreciate that the dosage regime or therapeutically effective amount of the compound to be administered may need to be optimized for each individual. In some embodiment the solutions are used to deliver a daily intravenous dose of about 0.01 mg/kg to 100 mg/kg body weight, typically between about 0.1 mg/kg and about 50 mg/kg body weight, may be appropriate, depending on the route and frequency of administration. The daily dose will typically be administered in one or multiple, e.g., two, three or four, doses per day.

The dose to be administered, for either treatment or prophylaxis, may be in the form of single bolus, infusion of the solution over a period of time, periodic dosing or a combination thereof. The concentration of any given dose will depend on the frequency of administration. In some embodiments, depending on the formulation, the dose of quetiapine for the treatment of delirium in an ICU patient will be approximately 10 mg/day, 15 mg/day, 20 mg/day, 25 mg/day, 30 mg/day, 35 mg/day, 40 mg/day, 45 mg/day, or about 50 mg/day.

Common side-effects associated with quetiapine include sedation, numbing, constipation, dizziness, dry mouth, lightheadedness, nasal congestion, sore throat, stomach pain or upset, tiredness, vomiting, weakness, weight gain, hyperlipidemia, hypotension, hyperglycemia and more. In one embodiment, the use of the solutions described herein results in elimination, amelioration, reduction, delay of onset or improvement in common side-effects associated with chronic or acute administration of quetiapine, wherein the common side-effects include but are not limited to constipation, dizziness, dry mouth, lightheadedness, nasal congestion, sore throat, stomach pain or upset, tiredness, vomiting, weakness, weight gain, hyperlipidemia, hypotension, hyperglycemia or their combination.

Quetiapine is useful for the treatment of and approved for schizophrenia, bipolar disorder, generalized anxiety disorder, dementia, obsessive compulsive disorder, acute manic episodes, psychotic symptoms associated with Parkinson's disease, as an adjunctive treatment for major depressive disorder, and in the treatment of delirium, particularly in patient an intensive care setting.

Delirium is an acute, reversible and fluctuating alteration in consciousness and mentation. It is reported in up to 33% of acutely hospitalised patients and in up to 80% of patients in intensive care. Delirium often manifests as a reduction in the level of consciousness with increased somnolence, decreased cognition and responsiveness or as a state of hypervigilance, agitation and confusion (hyperactive delirium). Delirium may also fluctuate between these two extremes. Delirium is also characterized by hallucinations and delusions, which can occur as the predominant and most distressing symptoms. In one embodiment the solutions described herein can be used to treat hallucinations and/or delusions associated with delirium.

Delirium has a significant impact upon patients within the ICU, and is associated with increased mortality, increased length of ICU and hospital stay, increased length of ventilator dependence and increased use of sedatives, which may be harmful. Delirium is also a significant risk factor for development of post-ICU cognitive dysfunction, including post-traumatic stress disorder.

Managing patients with delirium is extremely challenging, optimal management requires a calm environment, sleep, good hygiene, correction of underlying contributing factors and minimising sedative use. These can be challenging in the ICU due to the environment and patient's generally lacking normal levels of acuity. Agitated patients may harm themselves for example by inadvertently removing endotracheal tubes, venous access or invasive monitoring. Hence, to preserve patient safety, conventional drug therapy including haloperidol, clonidine, benzodiazepines or propofol is commonly used to manage delirium in an ICU setting.

Haloperidol is a standard drug therapy for ICU delirium, despite significant limitations due to adverse events, including QTc prolongation (and potential induction of ventricular tachycardia, especially Torsades de Pointes), hypotension and extrapyramidal side effects. However, haloperidol may prolong delirium in ICU patients and to ensure maximum effect and minimise adverse events, it is generally accepted that haloperidol should be administered in small, frequent doses for example 0.5 to 1 mg every two to four hours. This can be difficult to achieve in clinical practice and is a burden on clinical staff, thus poor adherence to recommended dosage regimes can contribute to delirium and agitation or result in an increase in adverse events.

Quetiapine has demonstrated efficacy in psychiatry and can be of benefit in the management of delirium but this is difficult when enteral administration is not possible. In addition, prior to the present invention quetiapine's insolubility prevented other modes of administration. Quetiapine has low binding at D2 receptors, which has been reported to be favorable in delirium management. In addition, quetiapine has several pharmacokinetic properties that make it potentially advantageous in the ICU including that it causes a lower incidence of QTc prolongation and fewer extrapyramidal symptoms than haloperidol.

Quetiapine may be administered along with a pharmaceutical carrier, diluent or excipient as described above. Alternatively, or in addition, quetiapine may be administered in combination with other agents, for example, other agents for treating delirium such as other atypical antipsychotics (e.g. clozapine, olanzapine, risperidone, ziprasidone), anticholinergic drugs (e.g. rivastigmine), alpha-2 agonists (e.g. clonidine), or benzodiazepine.

In some embodiments, quetiapine may be administered in combination with non- pharmacological interventions addressing delirium. The non-pharmacological interventions include environmental modification, for example the use of earplugs at bedtime leads to better sleep and delirium prevention, especially if used within 48 h of admission. Alternatively or in addition the environmental modification may include minimizing the level of noise by discontinuing the use of unnecessary monitors or equipment, adjusting alarm volumes to the safest minimum level.

The National Institute for Health and Care Excellence developed guidelines to improve sleep in which they recommend, where possible, avoiding intervention during sleeping hours, scheduling medication rounds to avoid sleep disruption, and minimizing noise during sleep time.

Critically ill patients requiring ventilation often receive sedation and analgesia to relieve pain and anxiety and thus reduce physical stress and oxygen consumption. An evidence-based approach used for weaning from ventilation is the Awakening and Breathing Coordination of daily sedation and ventilator removal trials, Choice of sedative or analgesic exposure, Delirium monitoring and management, and Early mobility and Exercise (ABCDE) bundle. The ABCDE bundle can be adopted for some patients on a daily basis (unless stopped by the treating physician). The “wake up and breathe” protocol combines daily interruption of sedation with daily spontaneous breathing trials can also be used.

The terms “combination therapy” or “adjunct therapy” in defining use of the quetiapine solutions described herein and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

A number of agents are available in commercial use, in clinical evaluation and in pre-clinical development, which could be selected for treatment of approved for schizophrenia, acute manic episodes, and adjunctive treatment for major depressive disorder as part of combination drug therapy. Suitable agents which may be used in combination therapy will be recognized by those of skill in the art. Suitable agents are listed, for example, in the Merck Index, An Encyclopaedia of Chemicals, Drugs and Biologicals, 12th Ed., 1996, and subsequent editions, the entire contents of which are incorporated herein by reference.

Combination regimens may involve the active agents being administered together, sequentially, or spaced apart as appropriate in each case. Combinations of active agents including compounds of the invention may be synergistic.

The co-administration of quetiapine solutions described herein may be effected by the quetiapine being in the same unit dose as another active agent, or the quetiapine and one or more other active agent(s) may be present in individual and discrete unit doses administered at the same, or at a similar time, or at different times according to a dosing regimen or schedule. Sequential administration may be in any order as required, and may require an ongoing physiological effect of the first or initial compound to be current when the second or later compound is administered, especially where a cumulative or synergistic effect is desired.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

In order that the present technology may be more clearly understood, preferred embodiments will be described with reference to the following drawings and examples.

### EXAMPLES

The present inventors prepared a number of aqueous quetiapine solutions by combining quetiapine and the excipients as set out in Table 1, below.

TABLE 1
Quetiapine solutions

| Formula #1 (5 mg/ml) | Formula #2 (5 mg/ml) |
|---|---|
| Quetiapine Fumarate (5.76 mg) | Quetiapine Fumarate (5.76 mg) |
| Sodium Benzoate (24.5 mg) | Sodium Benzoate (46.8 mg) |
| Benzoic acid (0.5 mg) | Benzoic acid (0.96 mg) |
| L-Arginine (1.99 mg) | L-Arginine (1.3 mg) |
| Propylene Glycol (200 mg) | Propylene Glycol (200 mg) |
| WFI qs 1 mL | WFI qs 1 mL |
| pH 6.60 | pH 6.52 |
| Formula #3 (5 mg/ml) | Formula #4 (5 mg/ml) |
| Quetiapine Fumarate (5.76 mg) | Quetiapine Fumarate (5.76 mg) |
| Sodium Benzoate (46.8 mg) | Sodium Benzoate (288 mg) |
| Benzoic acid (0.96 mg) | Benzoic Acid (1 mg) |
| L-Arginine (1.3 mg) | WFI qs 1 ml |
| Propylene Glycol (100 mg) | pH 6.81 |
| WFI qs 1 mL | |
| pH 6.32 | |
| Formula #5 (5 mg/ml) | Formula #6 (5 mg/ml) |
| Quetiapine Fumarate (5.76 mg) | Quetiapine Fumarate (5.76 mg) |
| Sodium Benzoate (125 mg) | Sodium Benzoate (144 mg) |
| WFI qs 1 ml | Benzoic Acid (0.5 mg) |
| pH 6.57 | WFI qs 1 ml |
| | pH 6.44 |
| Formula #7 (0.5 mg/ml) | Formula #8 (1 mg/ml) |
| Quetiapine Fumarate (0.576 mg) | Quetiapine Fumarate (1.152 mg) |
| Sodium Benzoate (41 mg) | Sodium Benzoate (41 mg) |
| WFI qs 1 ml | WFI qs 1 ml |
| pH 6.80 | pH 6.80 |
| Formula #9 (0.5 mg/ml) | Formula #10 (1 mg/ml) |
| Quetiapine Fumarate (0.576 mg) | Quetiapine Fumarate (1.152 mg) |
| Sodium Citrate (0.75 mg) | Sodium Citrate (0.75 mg) |
| Sodium Chloride (8.5 mg) | Sodium Chloride (8.5 mg) |
| WFI qs 1 mL | WFI qs 1 mL |
| pH 6.5 | pH 6.3 |

Formula #1: Quetiapine fumarate (QF) is added to propylene glycol with heating 70° C. and mixing in one beaker. In a separate beaker, benzoic acid and sodium benzoate are dissolved. This solution is then added to the first beaker with QF and propylene glycol and mixed for 30 mins. The solution is cooled to room temperature and L-Arginine is added to a resultant pH of 6.60. Sterile filtered with 0.2 μm cellulose filter into amber type 1 glass vial with rubber bung and aluminium seal. Autoclaved 121° C., 15 mins.

Formula #2: Quetiapine fumarate (QF) is added to propylene glycol with heating 70° C. and mixing in one beaker. In a separate beaker, benzoic acid and sodium benzoate are dissolved. This solution is then added to the first beaker with QF and propylene glycol and mixed for 30 mins. The solution is cooled to room temperature and L-Arginine is added to a resultant pH of 6.52. Sterile filtered with 0.2 μm cellulose filter into amber type 1 glass vial with rubber bung and aluminium seal. Autoclaved 121° C., 15 mins.

Formula #3: Quetiapine fumarate (QF) is added to propylene glycol with heating 70° C. and mixing in one beaker. In a separate beaker, benzoic acid and sodium benzoate are dissolved. This solution is then added to the first beaker with QF and propylene glycol and mixed for 30 mins. The solution is cooled to room temperature and L-Arginine is added to a resultant pH of 6.32. Sterile filtered with 0.2 μm cellulose filter into amber type 1 glass vial with rubber bung and aluminium seal. Autoclaved 121° C., 15 mins.

Formula #4: Sodium benzoate and benzoic acid are weighed and dissolved in WFI with the aid of heat at 70° C. and mixing in one beaker. Quetiapine fumarate (QF) is slowly added to this solution and mixed until dissolved. The solution is then cooled down and made up to volume with more WFI. Resultant pH is 6.81. Sterile filtered with 0.2 μm cellulose filter into amber type 1 glass vial with rubber bung and aluminium seal. Autoclaved 121° C., 15 mins.

Formula #5: Sodium benzoate is weighed and dissolved in WFI with the aid of heat at 70° C. and mixing in one beaker. Quetiapine fumarate (QF) is slowly added to this solution and mixed until dissolved. The solution is then cooled down and made up to volume with more WFI. Resultant pH is 6.57. Sterile filtered with 0.2 μm cellulose filter into amber type 1 glass vial with rubber bung and aluminium seal. Autoclaved 121° C., 15 mins.

Formula #6: Sodium benzoate and benzoic acid are weighed and dissolved in WFI with the aid of heat at 70° C. and mixing in one beaker. Quetiapine fumarate (QF) is slowly added to this solution and mixed until dissolved. The solution is then cooled down and made up to volume with more WFI. Resultant pH is 6.44. Sterile filtered with 0.2 μm cellulose filter into amber type 1 glass vial with rubber bung and aluminium seal. Autoclaved 121° C., 15 mins.

Formula #7: Sodium benzoate is weighed and dissolved in WFI with mixing in one beaker. Quetiapine fumarate (QF) is slowly added to this solution and mixed until dissolved. The solution is made up to volume with more WFI. Resultant pH is 6.80. Sterile filtered with 0.2 μm cellulose filter into amber type 1 glass vial with rubber bung and aluminium seal. Autoclaved 121° C., 15 mins.

Formula #8: Sodium benzoate is weighed and dissolved in WFI with mixing in one beaker. Quetiapine fumarate (QF) is slowly added to this solution and mixed until dissolved. The solution is made up to volume with more WFI. Resultant pH is 6.80. Sterile filtered with 0.2 μm cellulose filter into amber type 1 glass vial with rubber bung and aluminium seal. Autoclaved 121° C., 15 mins.

Formula #9: Sodium citrate, sodium chloride and Quetiapine fumarate (QF) are weighed and dissolved in WFI with the aid of heat at 70° C. and mixing in one beaker and mixed until dissolved. The solution is then cooled down and made up to volume with more WFI. Resultant pH is 6.50. Sterile filtered with 0.2 μm cellulose filter into amber type 1 glass vial with rubber bung and aluminium seal. Autoclaved 121° C., 15 mins.

Formula #10: Sodium citrate, sodium chloride and Quetiapine fumarate (QF) are weighed and dissolved in WFI with the aid of heat at 70° C. and mixing in one beaker and mixed until dissolved. The solution is then cooled down and made up to volume with more WFI. Resultant pH is 6.30. Sterile filtered with 0.2 μm cellulose filter into amber type 1 glass vial with rubber bung and aluminium seal. Autoclaved 121° C., 15 mins.

1. An aqueous solution essentially of water, a quetiapine salt and either or both of a benzoic acid salt and a citric acid salt, wherein the pH of the solution is in the range of 5-7.

2. The solution of claim 1 wherein the concentration of the quetiapine salt is at least 0.1 mg/ml.

3. The solution of claim 1, wherein the concentration of the quetiapine salt is at least about 0.1 mg/ml to about 5 mg/ml.

4. The solution claim 1, wherein the quetiapine salt is selected from the group consisting of quetiapine fumarate, quetiapine hemifumarate, quetiapine hydrobromide, quetiapine hydrochloride, quetiapine malate, quetiapine tartrate, quetiapine oxalate, quetiapine succinate, quetiapine benzoate, quetiapine sulfonate, quetiapine naphthalene-sulfonate, quetiapine naphsylate, and quetiapine tosylate.

5. The solution of claim 4 wherein the quetiapine salt is quetiapine fumarate.

6. The solution of claim 1, wherein the quetiapine is not conjugated to an amino acid or fatty acid.

7. The solution of claim 1, wherein the benzoic acid salt is selected from the group consisting of sodium benzoate, potassium benzoate, calcium benzoate, magnesium benzoate, lithium benzoate, and ammonium benzoate.

8. (canceled)

9. The solution of claim 1, wherein the citric acid salt is selected from the group consisting of calcium citrate, sodium citrate, and potassium citrate.

10. (canceled)

11. The solution of claim 1, further comprising one or more of an amino acid, a glycol and a pharmaceutically acceptable organic acid.

12. The solution of claim 11, wherein the amino acid is an L-amino acid selected from the group consisting of alanine, arginine, asparagine, aspartate, cysteine, glutamate, glutamine, glycine, histidine, hydroxyproline, isoleucine, leucine, lysine, methionine, phenylalanine, proline, pyroglutamate, serine, threonine, tryptophan, tyrosine, and valine.

13. The solution of claim 12, wherein the amino acid is L-arginine.

14. The solution of claim 11 wherein the pharmaceutically acceptable organic acid is one or more of benzoic acid, citric acid, oxalic acid, malic acid, tartaric acid, lactic acid, ascorbic acid, and glucuronic acid.

15. (canceled)

16. The solution of claim 11 wherein the glycol is selected from the group consisting of propylene glycol, ethylene glycol, polypropylene glycol, and polyethylene glycol.

17. (canceled)

18. The solution of claim 1, further comprising one or more pharmaceutically acceptable carriers, diluents or excipients.

19. The solution of claim 1 formulated for intravenous use.

20. The solution of claim 1 wherein the pH is about 6.1, 6.2, 6.3, 6.4, 6.5, 6.6, 6.7, 6,8, 6.9 or about 7.0.

21. A method of preventing, treating or ameliorating at least one symptom of delirium in a subject the method comprising administering to the subject a therapeutically effective amount of the aqueous solution of claim 1.

22. The method of claim 21 wherein the delirium is characterized by a state of reduced cognition and responsiveness; a state of hypervigilance, agitation and confusion; or a fluctuation between the states.

23. The method of claim 21 wherein the subject is critically ill.

24. The method of claim 21 wherein the administering step comprises intravenous administration or subcutaneous administration of the aqueous solution.

25. (canceled)
